(19) United States
(12) Patent Application Publication
(10) Pub. No.: US 2026/0335618 A1
(43) Pub. Date: Oct. 29, 2026

(54) HANDHELD PRECISION MICROBLADING PRINTER

(71) Applicant: L'Oreal, Paris (FR)

(72) Inventor: Mick Anthony YOON, Brooklyn, NY (US)

(73) Assignee: L'Oreal, Paris (FR)

(21) Appl. No.: 19/193,358

(22) Filed: Apr. 29, 2025

(51) Int. Cl. A61M 37/00

(52) U.S. Cl. CPC A61M 37/0076; A61M 37/0015; A61M 2037/0007; A61M 2037/0061; A61M 2205/3344

(57) ABSTRACT

A microblading device, including at least one position sensor configured to measure a position of the microblading device, one or more reservoirs, each configured to hold one or more inks, an applicator configured to apply the one or more inks into skin, a power source, at least one pressure sensor configured to measure a pressure of the applicator, and a processor configured to receive an image file comprising an eyebrow cosmetic style, detect the position and a curvature of a body based on the position sensor, direct the applicator to apply the eyebrow cosmetic style in a specific location with the one or more inks, and modulate the pressure of the applicator based on the image file.

### SUMMARY

In one aspect, disclosed herein is a microblading device, including at least one position sensor configured to measure a position of the microblading device. one or more reservoirs, each configured to hold one or more inks, an applicator configured to apply the one or more inks into skin, a power source, at least one pressure sensor configured to measure a pressure of the applicator, and a processor configured to receive an image file comprising an eyebrow cosmetic style, detect the position and a curvature of a body based on the position sensor, direct the applicator to apply the eyebrow cosmetic style in a specific location with the one or more inks, and modulate the pressure of the applicator based on the image file.

In some embodiments, the applicator is one or more microneedles. In some embodiments, wherein the one or more microneedles are arranged in an array. In some embodiments, the device further comprises a motor configured to reciprocate the microneedle.

In some embodiments, the applicator is one or more electrodes configured to apply the one or more inks with electroporation. In some embodiments, the microblading device further comprises a pulse generator configured to deliver an electrical current through the one or more electrodes.

In some embodiments, the at least one position sensor is configured to detect a plane normal to the body, and wherein the processor is further configured to prevent the applicator from applying the eyebrow cosmetic style when an angle between the applicator and the plane normal to the body exceeds an angular threshold.

In some embodiments, the processor is further configured to modulate a penetration depth of the applicator based on the eyebrow cosmetic style. In some embodiments, the penetration depth ranges from about 0.2 mm to about 2 mm into skin.

In another aspect, disclosed herein is a system for applying cosmetics, including the microblading device described herein and a smart device comprising an application configured to select the eyebrow cosmetic style from a plurality of eyebrow cosmetic styles, display the eyebrow cosmetic style on an image of a body, and transmit the eyebrow cosmetic style as an image file to the microblading device.

In some embodiments, the image file further comprises one or more pressure metrics, one or more penetration depths, or a combination thereof. In some embodiments, the eyebrow cosmetic style comprises one or more eyebrow mimetics. In some embodiments, the one or more eyebrow mimetics includes a randomization factor, one or more hair-like strokes, one or more fractals, or a combination thereof.

In some embodiments, the application is further configured to display one or more splines of the eyebrow cosmetic style on the image of the body.

In yet another aspect, disclosed herein is a method of using a microblading device, the microblading device comprising at least one position sensor, one or more reservoirs, each configured to hold one or more inks, an applicator configured to apply the one or more inks, at least one pressure sensor, a power source; and a processor, the method including selecting an eyebrow cosmetic style from a plurality of eyebrow cosmetic styles, displaying the eyebrow cosmetic style on an image of a body, transferring the eyebrow cosmetic style as an image file to the microblading device, wherein the image file further comprises one or more pressure metrics, moving the microblading device over the body, applying the eyebrow cosmetic style onto the body at a specific location based on one or more position sensors on the microblading device, and modulating a pressure of the applicator based on the one or more pressure metrics.

In some embodiments, the method further includes recommending the eyebrow cosmetic style based on a trending eyebrow cosmetic style, a color of a user's hair, skin, or lips, a past eyebrow cosmetic style selected by the user, a shape of the user's eyes, eyebrows, nose, lips, cheeks, or forehead, a location of the user, a color of the user's clothing or a combination thereof.

In some embodiments, the method further includes adjusting the eyebrow cosmetic style, wherein adjusting the eyebrow cosmetic style comprises changing a color, a size, an angle, a length, a width, a hair size, a pattern, a position, a location of the eyebrow cosmetic style, or a combination thereof.

In some embodiments, the microblading device applies the eyebrow cosmetic style with one or more microneedles, one or more electrodes, or a combination thereof.

In some embodiments, the method further includes detecting a plane normal to the body with the one or more position sensors and preventing the applicator from applying the eyebrow cosmetic style when an angle between the applicator and the plane normal to the body exceeds an angular threshold.

In some embodiments, the method further includes displaying one or more splines of the eyebrow cosmetic style on the image of the body and adjusting the one or more splines in response to user input.

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

### DESCRIPTION OF THE DRAWINGS

FIG. 1A is an example front side of a microblading device, in accordance with the present technology;

FIG. 1B is an example back side of a microblading device, in accordance with the present technology;

FIG. 2A is an example exploded front side of a microblading device, in accordance with the present technology;

FIG. 2B is an example exploded back side of a microblading device, in accordance with the present technology;

FIG. 2C shows example internal components of a microblading device, in accordance with the present technology;

FIG. 2D shows another example of internal components of a microblading device, in accordance with the present technology;

FIG. 3 is an example system for autonomously applying a eyebrow cosmetic style, in accordance with the present technology;

FIGS. 4A-4D are example applicators, in accordance with the present technology;

FIGS. 5A-5B show an example user interface of an example microblading device, in accordance with the present technology;

FIGS. 6A-6C are example process diagrams of an example system in use, in accordance with the present technology;

FIGS. 7A-7E are example process diagrams of an example system in use, in accordance with the present technology;

FIG. 8 is an example method of using an example system, in accordance with the present technology; and

FIG. 9 is another example method of using an example system, in accordance with the present technology.

The foregoing aspects and many of the attendant advantages of this invention will become more readily appreciated as the same become better understood by reference to the following detailed description, when taken in conjunction with the accompanying drawings.

### DETAILED DESCRIPTION

Disclosed herein are devices, systems, and methods for at home, personalized, and custom microblading. As described herein, the term microblading may include both the use of microneedles configured to apply one or more inks or dyes into the skin and/or electrodes configured to apply one or more inks or dyes into the skin with electroporation. In some embodiments, a user may select, adjust, and create cosmetic styles, including eyebrows, eyeliner, concealer, freckle, and lip cosmetic styles, and apply these cosmetic styles to their skin. In some embodiments, the device is configured to detect a pressure of the applicator of the device and modulate a pressure or penetration depth of the applicator. In some embodiments, a position sensor of the microblading device may detect a plane normal to the user's skin or face and prevent the applicator from applying the cosmetic style when an angle between the user's skin or face and the applicator exceeds an angular threshold.

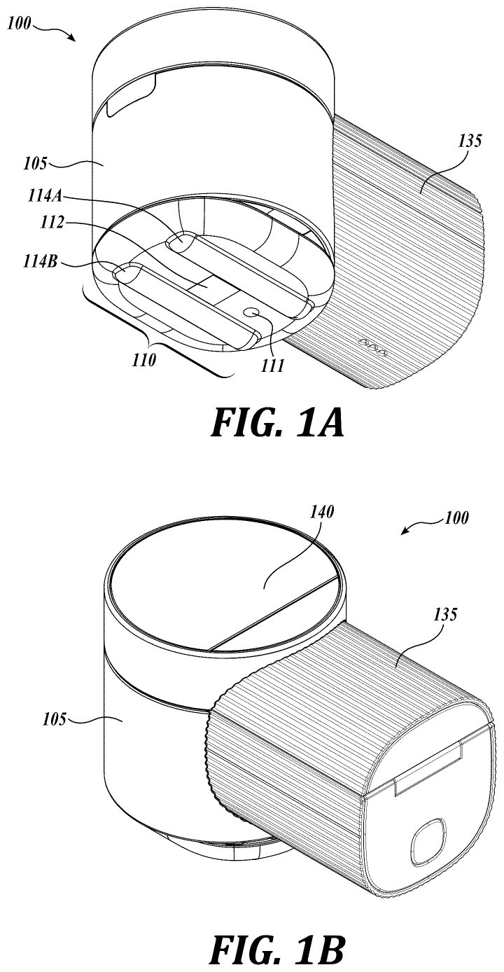

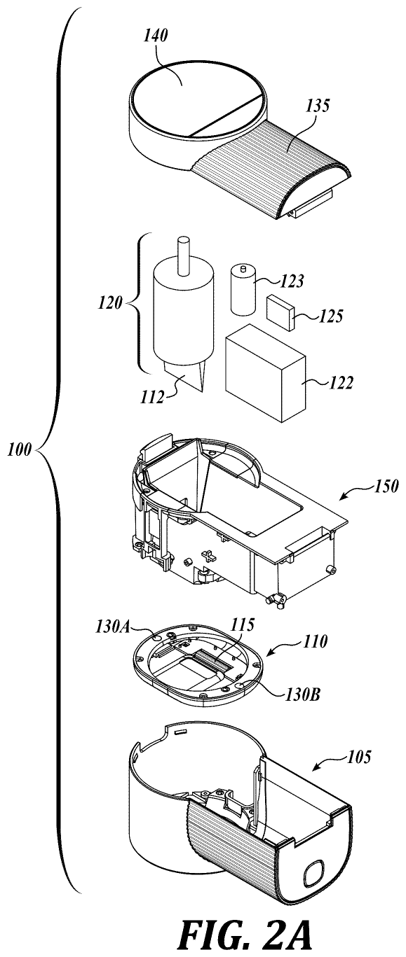

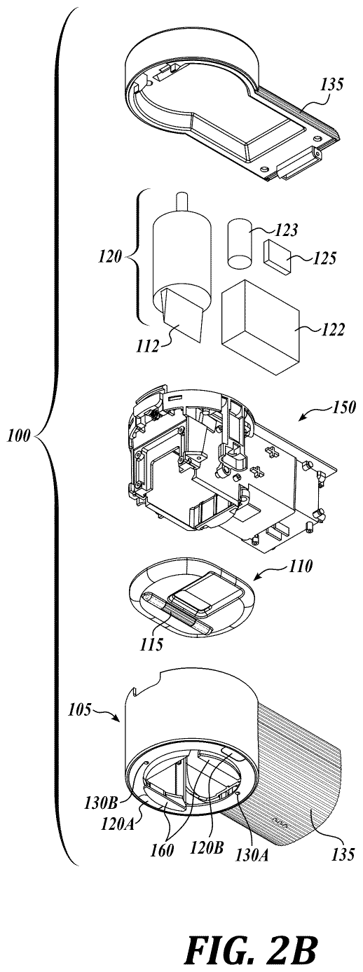

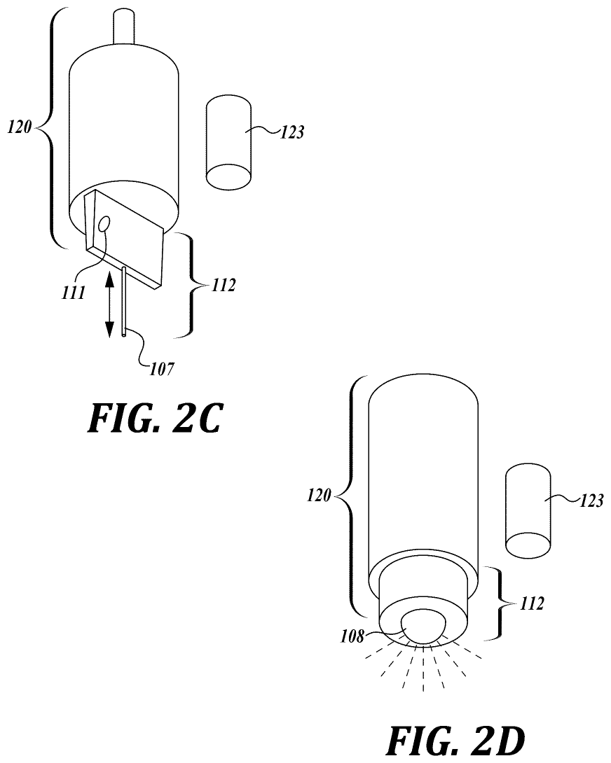

FIG. 1A is an example front side of a microblading device 100, in accordance with the present technology. In some embodiments, the microblading device 100 includes a body 105, and a handle 135. While microblading device 100 is illustrated with a cylindrical body 105 and a cylindrical handle 135, it should be understood that the microblading device 100 can take any number of forms. In some embodiments, the microblading device 100 does not have a handle 135. In some embodiments, the microblading device 100 includes internal circuitry, including a processor (such as processor 125), a battery (such as power source 123), and the like, as shown and described in FIGS. 2A-2D.

In some embodiments, the microblading device 100 includes a processor (as shown in FIG. 2A) configured to receive an image file, detect a position and a curvature of the body based on the position sensor, and direct an applicator 112 to apply an eyebrow cosmetic style based on the image file in a specific location on a surface, such as a user's face. In some embodiments, the specific location is determined by the eyebrow cosmetic style. For example, an eyebrow cosmetic style may be applied on the eyebrows of a user. In some embodiments, the image file may include one or more pressure metrics, one or more penetration depths, or a combination thereof. In such embodiments, the image file may provide instructions to the processor to modulate a pressure or penetration depth depending on which portion of the cosmetic style is being applied. For example, in areas of the cosmetic style intended to be darker, the pressure may be increased. As another example, in areas of the cosmetic style intended to be less permanent, the penetration depth may be reduced.

In some embodiments, the microblading device 100 is powered through a wired connection, but the microblading device 100 may also be independently powered, such as with a battery or a capacitor (shown as power source 123). In some embodiments, the microblading device 100 may further include a charging port, configured to power the microblading device 100.

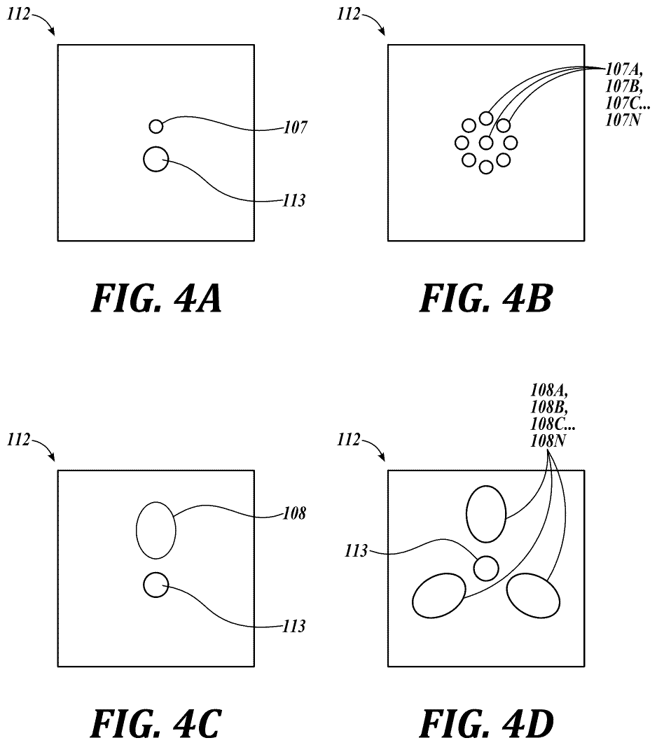

In some embodiments, the body 105 houses an applicator 112. In some embodiments, the applicator 112 is positioned on a front side 110 of the microblading device 100, such as shown in FIG. 1A. In some embodiments, the applicator 112 includes one or more microneedles, one or more electrodes, or a combination thereof (as shown and described in FIGS. 4A-4D).

In some embodiments, the applicator 112 is configured to facilitate apply a cosmetic style (such as an eyebrow cosmetic style) onto a surface (such as skin). In some embodiments, the applicator 112 is rectangular, square, circular, organically shaped, or the like. In some embodiments, the applicator 112 is in the middle of front side 110 of the body 105. In some embodiments, the applicator 112 is located between spacers 114A, 114B.

While two spacers 114A and 114B are illustrated, it should be understood that any number of spacers 114A, 114B may be on the front side 110 of the microblading device 100. In some embodiments, the spacers 114A, 114B are rounded polygons, such as shown in FIG. 1A, but it should be understood that the spacers 114A, 114B can take any number of forms including spherical, rectangular, and organically shaped. In some embodiments, the spacers 114A, 114B are configured to contact a surface while the microblading device 100 is passed over it, so that an optimal distance between the applicator 112 and the surface is achieved. In some embodiments, the spaces 114A and 114B have a thickness that allows the printer applicator 112 to be in contact with a curved surface. In some embodiments, the spacers 114A and 114B may be configured to roll. In some embodiments, the spacers 114A and 114B may include at least one position sensor, as described herein. In some embodiments, in addition to maintaining a distance between the applicator 112 and the surface, the spacers 114A and 114B are configured to roll on the surface as the microblading device 110 applies the cosmetic style.

In some embodiments, the microblading device 100 includes at least one position sensor. (as shown and described in FIG. 2A). In some embodiments, the position sensor may be housed inside the body 105, but in some embodiments, the position sensor may be located on the front side 110 of the microblading device 100. In some embodiments, the at least one position sensor may be inside the spacers 114A, 114B, or a combination thereof. In some embodiments, the microblading device 100 further includes a camera (as shown and described in FIG. 2A). In some embodiments, the camera is configured to take a plurality of images as the printer 110 moves over a facial feature. In some embodiments, the facial feature may be an eyebrow, a nose, an eye, a wrinkle, acne, or the like.

In some embodiments, the front side 110 of the microblading device 100 is rotatably adjustable. In some embodiments, the front side 110 is configured to articulate to more accurately scan a surface, such as a body, skin, or hair. In such embodiments, the position sensor may be a sensor wheel as described herein. In operation, the position sensor contacts the surface and rolls as the microblading device 100 scans the surface. In such embodiments, the microblading device 100 is able to take into account the curvature of the surface. In some embodiments, the surface is a body of a user. In some embodiments, the front side 110 can be adjusted to fit the needs of different body types and printing environments. In some embodiments, the spacers 114A and 114B may be moved or adjusted. In some embodiments, the front side 110 may be concave or convex to better contact the surface. In some embodiments, the applicator 112 may similarly be concave or convex to better contact the surface. In some embodiments, the front side 110 is capable of being articulated, so as to better contact the surface. In some embodiments, the front side 110 is coupled to the device 100 with a flexible connector (as shown and described in FIG. 2B). In some embodiments, the flexible connector is a pivot, a hinge, or a joint. In some embodiments, the flexible connector allows the front side 110 to be articulated. In some embodiments, this allows for more accurate scans of a surface. In some embodiments, this further allows the front side 110 to determine a curvature of a surface (such as with the one or more position sensors).

In some embodiments, the microblading device 100 further includes one or more pressure sensors 111. In some embodiments, the one or more pressure sensors 111 may be located on the front side 110 of the microblading device 100. In other embodiments, the one or more pressure sensors 111 may be incorporated onto or into the applicator 112, and/or one or more internal components of the microblading device 100 as shown and described in FIGS. 2A-2D. In some embodiments, the one or more pressure sensors are configured to sense a pressure of the applicator 112 against the surface (such as skin). In some embodiments, the sensed pressure may be transmitted to a processor. The processor may then adjust the pressure of the applicator 112. In some embodiments, the pressure of the applicator 112 may be adjusted based on one or more pressure metrics of the makeup image file.

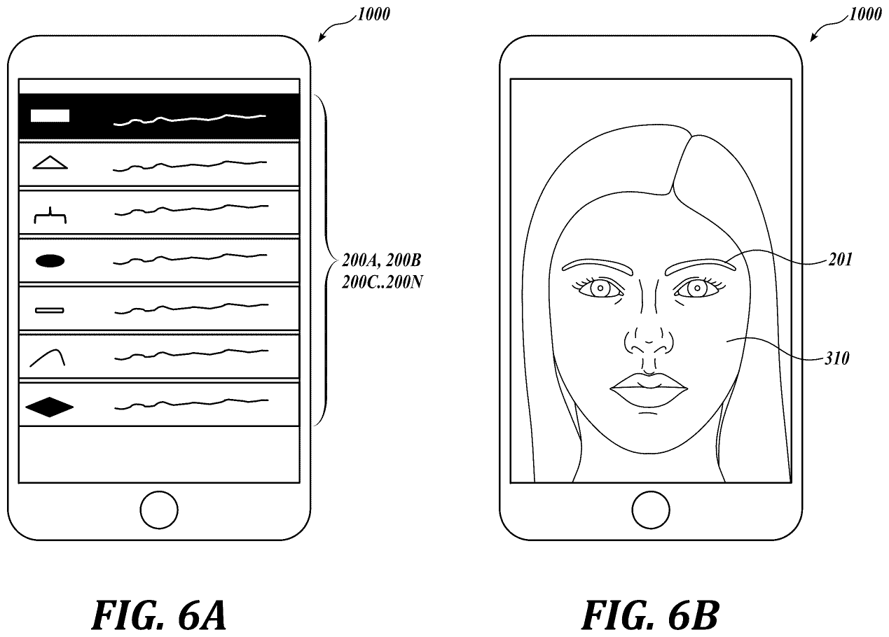

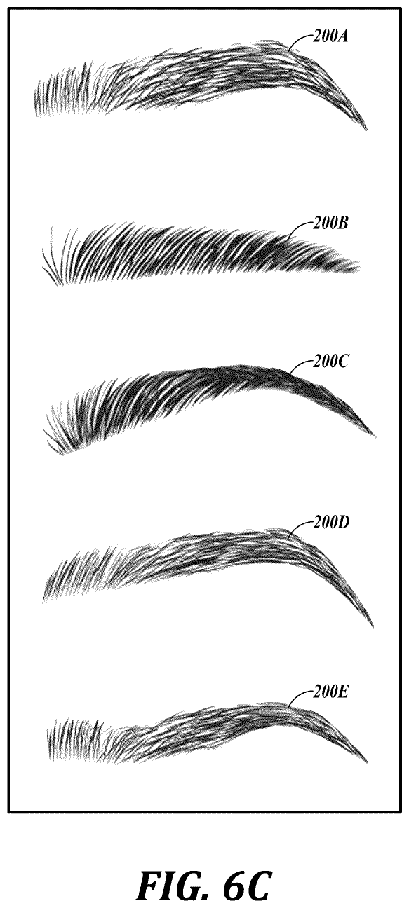

In some embodiments, if the sensed pressure exceeds a pressure threshold, the microblading device 100 turns off or prevents the applicator 112 from applying the cosmetic style. In some embodiments, the processor, based on the sensed pressure, is further configured to modulate a penetration depth of the applicator based on the eyebrow cosmetic style. In some embodiments, the penetration depth ranges from about 0.2 mm to about 2 mm into skin. The penetration depth may be based on a desired outcome of the user. For example, ink applied at a penetration depth of between about 0.2 mm to about 1.5 mm may be less permanent. Ink applied at a penetration depth between about 1.5 mm to about 2 mm may result in a darker, more permanent cosmetic style. Accordingly, the penetration depth may be adjusted depending on how permanent the user would like the cosmetic style and/or to achieve a darker or lighter ink deposition into the skin. Accordingly, some eyebrow cosmetic styles (as shown in FIG. 6C) may have multiple penetration depth metrics, such that the penetration depth is adjusted at different points in the eyebrow cosmetic style.

Similarly, in some embodiments, the at least one position sensor is configured to detect a plane normal to the body, and the processor is configured to prevent the applicator 112 from applying the eyebrow cosmetic style when an angle between the applicator 112 and the plane normal to the body exceeds an angular threshold.

FIG. 1B is an example back side of a microblading device 100, in accordance with the present technology. In some embodiments, the microblading device 100 further includes a user interface 140. Though the user interface 140 is illustrated on the backside of the microblading device 100, in some embodiments, the user interface 140 is a separate component, such as a smartphone or tablet. In some embodiments, the user interface 140 is round, but in other embodiments, the user interface 140 may take any form such as rectangular or oblong. In some embodiments, the user interface 140 includes one or more actuators, such as buttons or keys. In some embodiments, the user interface 140 includes a touch type capacitance button. In some embodiments, the user interface 140 is a touchscreen. In some embodiments, the user interface includes one or more output modules configured to output an alert. In some embodiments, the alert is a sound, vibration, or the like. In some embodiments, the alert includes an indication on how or in what direction to move the microblading device 100.

FIG. 2A is an example exploded front side of a microblading device 100, in accordance with the present technology, and FIG. 2B is an example exploded back side of a microblading device 100, in accordance with the present technology. In some embodiments, the microblading device 100 includes an internal component 150, a front side 110, and a position sensor 115. In some embodiments, the microblading device 100 further includes a reservoir 122, a processor 125, a power source 123, and a motor 120, coupled with an applicator 112.

In some embodiments, the internal component 150 is configured to hold the front side 110 in place. In some embodiments, the internal component 150 is coupled to the front side 110, the reservoir 122, the processor 125, the power source 123, and/or the motor 120.

In some embodiments, the front side 110 includes a positioning sensor 115 and at least one camera 130A, 130B. In some embodiments, the cameras 130A, 130B are located on the front side 110 but in some embodiments, the cameras 130A, 130B are located on the body 105. In some embodiments, as the applicator 112 moves across a surface, such as the user's face, the cameras 130A, 130B capture a plurality of images of the surface. In some embodiments, the cameras 130A, 130B take a plurality of images of a facial feature as the microblading device 100 moves over the facial feature. In some embodiments, the microblading device 100 includes two cameras 130A and 130B. In some embodiments, such as illustrated in FIG. 2A, the first camera 130B is located on a first side of the microblading device 100, and the second camera 130B is located on a second side of the microblading device 100, opposite the first side.

In some embodiments, the front side 110 includes one or more position sensors 115. While a single position sensor 115 is illustrated in FIG. 2A, it should be understood that any number of position sensors 115 may be utilized. In some embodiments, at least one position sensor 115 is a rolling position sensor 115, such as a sensor wheel. In such embodiments, the position sensor 115 is configured to roll across the facial feature as the front side 110 is moved over the facial feature. In this manner, position sensor 115 may detect a position of the facial feature as the microblading device 100 moves over the facial feature. In some embodiments, the position sensor 115 is further configured to detect the curvature of the facial feature or the user's face.

In some embodiments, the microblading device 100 includes a processor 125. In some embodiments, the processor 125 is communicatively coupled to the front side 110, the position sensor 115, one or more pressure sensors (such as pressure sensor 111) and the cameras 130A, 130B. The processor 125 may be configured to receive a makeup image file, detect a position and a curvature of the body based on the position sensor, and direct the applicator to apply the eyebrow cosmetic style based on the image file in a specific location. While a single processor 125 is illustrated, it should be understood that any number of processors may be incorporated into the microblading device 100.

In some embodiments, the microblading device 100 includes one or more reservoirs 122. In some embodiments, the one or more reservoirs 122 are configured to hold one or more cosmetic inks or dyes. In some embodiments, the reservoirs hold any number of cosmetic inks or dyes needed to apply the eyebrow cosmetic style. In some embodiments, the one or more reservoirs 122 include a number of cartridges, such that a single reservoir 122 can hold any number of colors, compositions, finishes, or formulations of the cosmetic inks or dyes. In some embodiments, the inks may include cadmium sulfide, cadmium selenide, iron oxides, ferric hydrate (red ochre), mercury sulfide, azo compounds, quinacridone, or a combination thereof. In some embodiments, the one or more inks include a pigment, such as an iron oxide-based pigment, an organic pigment, a blend of iron oxide and organic pigments, metallic inks, or a combination thereof. In some embodiments, the one or more inks are configured to activate in response to light, ultraviolet light, pressure, or the like. In some embodiments, the one or more inks are one or more UV responsive inks.

In some embodiments, the processor 125 is further communicatively coupled to the reservoir 145 and the applicator 112. In some embodiments, the processor 125 directs the reservoir 145 and the applicator 112 to apply an eyebrow cosmetic style. In some embodiments, the eyebrow cosmetic style is selected from an eyebrow, an eyeshadow, a concealer, a primer, a foundation, a blush, a lipliner, a lipstick, a bronzer, an eyeliner, a freckle pattern, a facial hair, a hair design, such as facial hair or a hairline design, or a highlighter.

FIG. 2C shows example internal components of a microblading device (such as microblading device 100), in accordance with the present technology. In some embodiments, the applicator 112 is one or more microneedles 107. In some embodiments, the one or more microneedles 107 are arranged in an array, as shown in FIG. 4B. In some embodiments, the microblading device 100 further includes a motor 120 configured to reciprocate the one or more microneedle 107. In some embodiments, one or more pressure sensors 111 are located on or inside the applicator 112. The pressure sensor 111 may measure a pressure of the one more microneedles 107. In some embodiments, the pressure sensor 111 transmits the measured pressure to a processor (such as processor 125). The processor may modulate the pressure of the one or more microneedles. In some embodiments, the processor is configured to stop the one or more microneedles from applying the cosmetic style if the measured pressure exceeds a pressure threshold.

FIG. 2D shows another example of internal components of a microblading device (such as microblading device 100), in accordance with the present technology. In some embodiments, the applicator 112 is one or more electrodes 112. In operation, the one or more electrodes 112 are configured to apply the one or more inks with electroporation. It should be understood that as used herein, the microblading device (such as microblading device 100) may not include microneedles, and may instead apply ink solely with electroporation. In some embodiments, the microblading device may apply ink with both one or more microneedles and one or more electrodes 108. In some embodiments, the microblading device further includes a pulse generator 121 configured to deliver an electrical current to the one or more electrodes 108. In operation, the one or more electrodes 108 temporarily create pores in cell membranes of a user's skin with a series of electrical pulses delivered by the pulse generator 121. The pores created may allow molecules, such as ink molecules, to enter the cell, thereby applying the cosmetic style.

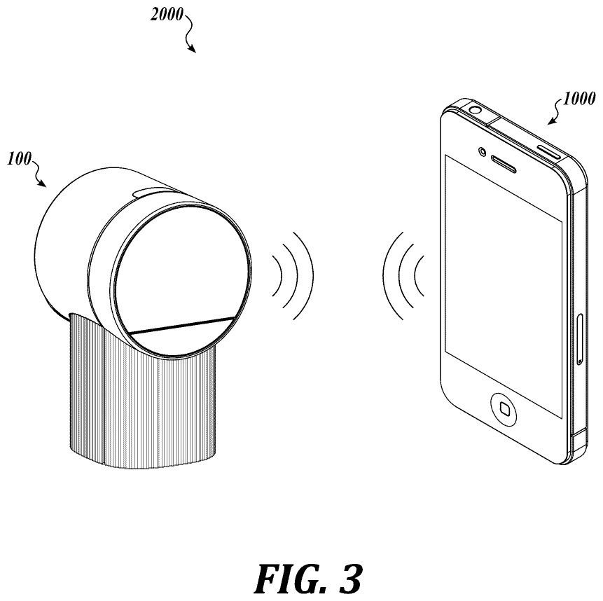

FIG. 3 is an example system 2000 for autonomously applying a eyebrow cosmetic style, in accordance with the present technology. Disclosed herein is a system 2000 for applying a eyebrow cosmetic style including a smart device 1000 and a microblading device100.

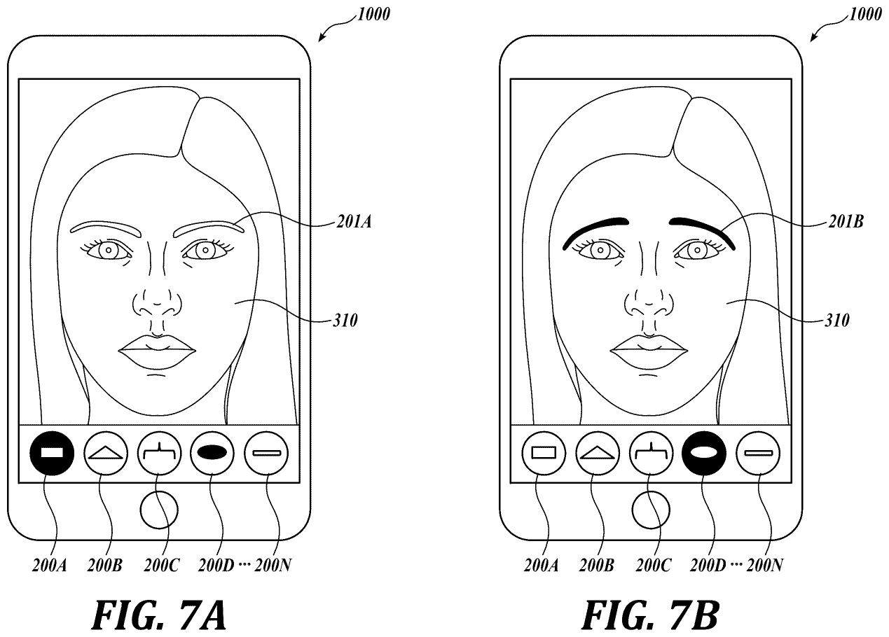

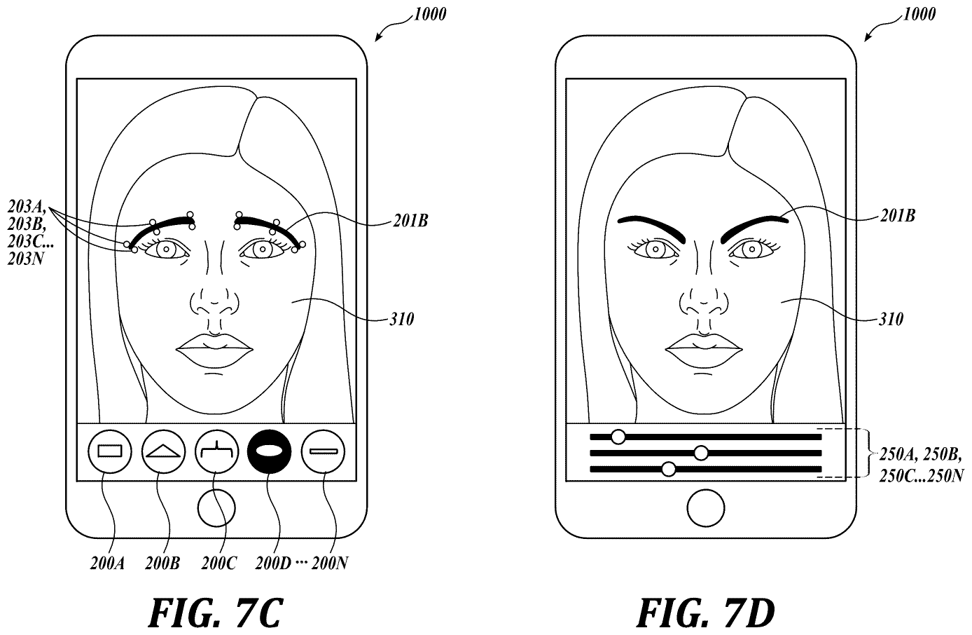

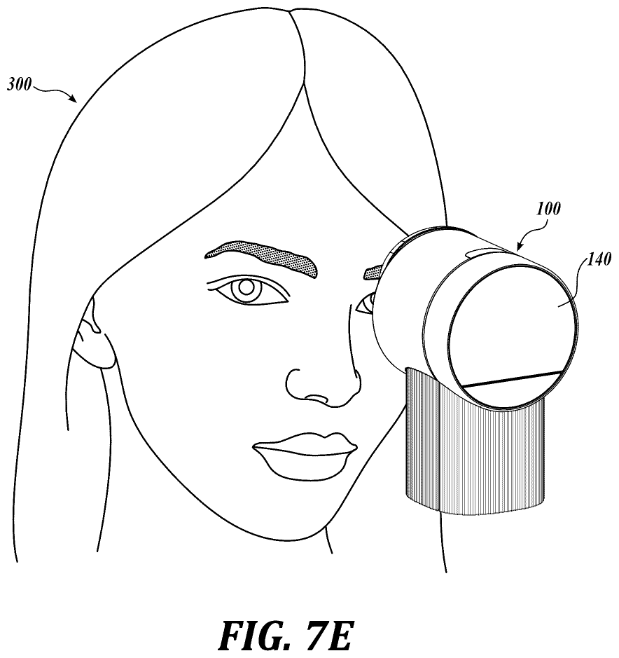

In some embodiments, the smart device 2000 includes an application configured to select the eyebrow cosmetic style from a plurality of eyebrow cosmetic styles, display the eyebrow cosmetic style on an image of a body, and transmit the eyebrow cosmetic style as a makeup image file, as shown and described herein in detail in FIGS. 6A-6B and 7A-7D. In some embodiments, such as illustrated in FIG. 3, the smart device 1000 is a smartphone. It should be understood that the smart device 1000 can take any number of forms, including a tablet, laptop, or computer. In some embodiments, the smart device 1000 is communicatively coupled to the microblading device100. In some embodiments, the smart device 1000 may be communicatively coupled to the microblading device100 by a wireless connection such as Bluetooth™, Bluetooth low energy (BLE)™, a Wi-Fi, Zigbee™, or wired connection.

In some embodiments, the microblading device 100 includes at least one position sensor, one or more reservoirs each configured to hold one or more dyes, and a printer including a printer applicator. In some embodiments, the printer is configured to print the eyebrow cosmetic style with the one or more dyes through the printer applicator. In some embodiments, the microblading device 100 further includes a processor configured to receive the makeup image file, detect a position and a curvature of the body based on the position sensor, and direct the printer to print the eyebrow cosmetic style in a specific location, as described herein.

FIGS. 4A-4D are example applicators 112, in accordance with the present technology. The applicators 112 illustrated in FIGS, 4A-4D are merely representative, and one skilled in the art will recognize that combinations of the applicators illustrated are within the scope of the present discloser.

FIG. 4A shows an example applicator 112 including a single microneedle 107 and an ink depositor 113. In some embodiments, the applicator 112 includes one or more microneedles, which may be a single microneedle 107 as illustrated in FIG. 4A. In some embodiments, the ink depositor 113 is fluidly coupled with a reservoir (such as reservoir 122). In operation, the ink depositor 113 may deposit the one or more inks in the reservoir and onto a surface, such as onto skin. The microneedle 107 may then apply the ink to the surface as its reciprocated over the skin. In some embodiments, the processor is further configured to modulate a penetration depth of the applicator based on the eyebrow cosmetic style. In some embodiments, the penetration depth ranges from about 0.2 mm to about 2 mm into skin.

FIG. 4B is an applicator 112 having an array of microneedles 107A 107B, 107C . . . 107N. In some embodiments, the array of microneedles 107A 107B, 107C. . . 107N is circular, but in other embodiments, the array of microneedles 107A 107B, 107C. . . 107N may be rectangular, square, organic, triangular, or the like. In some embodiments, one or more microneedles of the array of microneedles 107A 107B, 107C . . . 107N are hollow and fluidly coupled with the reservoir. In such embodiments, ink may be applied to the skin through one or more of the microneedles of the array of microneedles 107A 107B, 107C . . . 107N. In some embodiments, when the applicator 112 has a single microneedle 107, the single microneedle may be hollow, and the ink depositor 113 may be omitted.

FIG. 4C is an applicator 112 having a single electrode 108 and an ink depositor 113. In some embodiments, the applicator 112 includes one or more electrodes, which may be a single electrode 108 as illustrated in FIG. 4C. In some embodiments, the ink depositor 113 is fluidly coupled with a reservoir (such as reservoir 122). In operation, the ink depositor 113 may deposit the one or more inks in the reservoir and onto a surface, such as onto skin. The electrode 108 may then apply the ink to the surface as it delivers a series of pulses to the skin, through electroporation as described herein.

FIG. 4D is an applicator 112 having a plurality of electrodes 108A, 108B, 108C . . . 108N. While three electrodes are illustrated, it should be understood that any number of electrodes may be included on applicator 112.

In some embodiments, the applicator 112 may be removably coupled to the microblading device. In such embodiments, any of the applicators are shown in FIGS. 4A-4D may be utilized by a same microblading device.

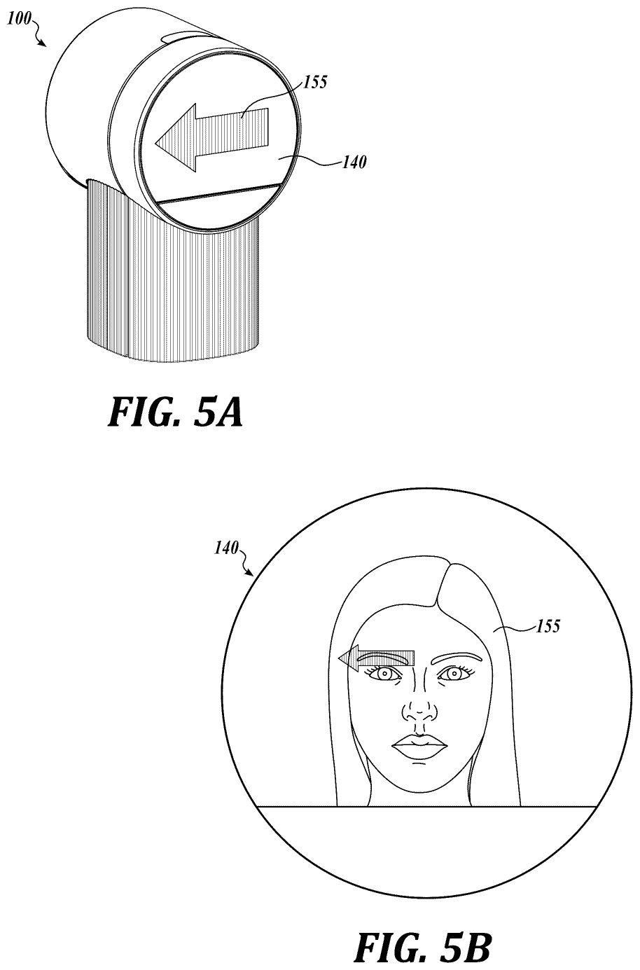

FIGS. 5A-5B show an example user interface 140 of an example microblading device 100, in accordance with the present technology.

In some embodiments, the user interface 140 is configured to display a microblading guide 155 in order to direct a user to properly use the microblading device 100. In some embodiments, the microblading guide 155 includes one or more of the plurality of images as described herein of a facial feature or surface and an arrow pointing in a direction a user can move the microblading device. In some embodiments, the microblading guide 155 includes a graphical representation of the facial feature 200 and an arrow pointing in the direction a user can move the microblading device.

In some embodiments, the user interface 140 displays a current view of one or more cameras on the microblading device 100. In some embodiments, as the user moves the microblading device 100 over the surface, an image captured by one or more cameras is displayed. In some embodiments, the microblading guide 155 further includes one or more alerts to direct the user to move the microblading device 100. In some embodiments, the alerts are visual alerts, such as arrows, auditory alerts, such as a chime or alarm, or tactile alerts such as vibrations.

FIGS. 6A-6B are example process diagrams of an example system in use, in accordance with the present technology. In some embodiments, the system may be system 2000 or system 2500 as described herein.

FIG. 6A is an example application on a smart device 1000. In some embodiments, the application is configured to provide a plurality of eyebrow cosmetic styles 200A, 200B, 200C . . .200N. It should be understood that eyebrow cosmetic styles are shown and described in FIGS. 6A-7E, but any of the cosmetic styles described herein may be applied by the microblading device 100, such as a lip cosmetic style, an eyeliner cosmetic style, a concealer cosmetic style, a freckle cosmetic style, or the like. In some embodiments, a user may select an eyebrow cosmetic style of the plurality of styles 200A, 200B, 200C . . . 200N. In some embodiments, the user may select the eyebrow cosmetic style by clicking, tapping, or otherwise choosing the eyebrow cosmetic style. In FIG. 6A, the selected eyebrow cosmetic style is shown in black. In some embodiments, the application may display the plurality of eyebrow cosmetic styles 200A, 200B, 200C . . . 200N as a list. In some embodiments, each eyebrow cosmetic style of the plurality of eyebrow cosmetic styles 200A, 200B, 200C . . . 200N includes a graphical representation of the style, a description of the style, or both. In some embodiments, the application is further configured to recommend the eyebrow cosmetic style from the plurality of eyebrow cosmetic styles 200A, 200B, 200C . . . 200N. In some embodiments, the recommendation is based on a trending eyebrow cosmetic style, a color of a user's hair, skin, or lips, a past eyebrow cosmetic style selected by the user, a shape of the user's eyes, eyebrows, nose, lips, cheeks, or forehead, a location of the user, or a color of the user's clothing.

FIG. 6B is an example application on the smart device 1000 displaying an overlay of the selected eyebrow cosmetic style 201 on an image of a user's face 310. In some embodiments, the image is a live video feed from a camera of the smart device 1000 of a camera of the microblading device (not shown in FIG. 6B). In some embodiments, the image is a static photo, or a previously taken video. In such embodiments, a user may upload a photo or a video to the application.

FIG. 6C shows a plurality of eyebrow cosmetic styles 200A, 200B, 200C, 200D, 200E in accordance with the present technology. In some embodiments, each eyebrow cosmetic style of the plurality of eyebrow cosmetic styles 200A, 200B, 200C, 200D, 200E may include or more eyebrow mimetics. In some embodiments, the one or more eyebrow mimetics includes a randomization factor, one or more hair-like strokes, one or more fractals, or a combination thereof. In some embodiments, the eyebrow mimetics are configured to provide an appearance of a natural eyebrow, or a creative eyebrow, such as an eyebrow including a slit, a pattern, or word, or the like.

FIGS. 7A-7D are example process diagrams of an example system in use, in accordance with the present technology. In some embodiments, the system may be system 2000 or system 2500 as described herein.

FIG. 7A is an example application on a smart device 1000. In some embodiments, FIG. 7A follows FIG. 6B. In some embodiments, a user may scroll through a plurality of eyebrow cosmetic styles 200A, 200B, 200C, 200D . . . 200N while displaying an image of the user's face 310 and the selected eyebrow cosmetic style 201A. In such embodiments, the user may select an eyebrow cosmetic style 210A and change between eyebrow cosmetic styles of the plurality of eyebrow cosmetic styles 200A, 200B, 200C, 200D . . . 200N in real time. In some embodiments, the plurality of eyebrow cosmetic styles 200A, 200B, 200C, 200D . . . 200N are shown in a manner that does not obscure the image of the user's face 310, such as at a bottom, top, or side of a screen of the smart device 1000. As shown in FIG. 7A, currently, eyebrow cosmetic style 200A has been selected, and is displayed as selected eyebrow cosmetic style 201A.

In FIG. 7B, a user has selected a new eyebrow cosmetic style 200D, which is displayed on the image of the user 310 as selected eyebrow cosmetic style 201B. In this manner, a user may change between any number of eyebrow cosmetic styles from the plurality of eyebrow cosmetic styles 200A, 200B, 200C, 200D . . . 200N before adjusting or printing a eyebrow cosmetic style.

FIG. 7C shows a plurality of splines 203A, 203B, 203C . . . 203N of the eyebrow cosmetic style 201B. In some embodiments, the user may adjust the eyebrow cosmetic style 201B by dragging or otherwise adjusting the one or more splines 203A, 203B, 203C . . . 203N of the eyebrow cosmetic style. In some embodiments, the one or more splines 203A, 203B, 203C . . . 203N can be toggled on and off. In some embodiments, a shape of the eyebrow cosmetic style 201B may be warped, adjusted, or moved with the one or more splines 203A, 203B, 203C . . . 203N.

FIG. 7D shows a user adjusting the selected eyebrow cosmetic style 201B to fit their preferences. In some embodiments, FIG. 7C follows FIG. 6B. In some embodiments, the application may suggest adjustments to the selected eyebrow cosmetic style 201B. In some embodiments, the suggested adjustments may be based on a shape of the user's face, the shape of a facial feature of the user, a color of the user's hair, skin, eyes, or clothing, or a trending adjustment. In some embodiments, adjusting the selected eyebrow cosmetic style 201B includes changing a color, a size, a length, a width, a hair size, a pattern, an angle, a position, a location of the eyebrow cosmetic style, or a combination thereof of the eyebrow cosmetic style 201B. In some embodiments, such as when the eyebrow cosmetic style 201B includes discrete elements, the discrete elements of the eyebrow cosmetic style 201B may be adjusted independently. For example, if the selected eyebrow cosmetic style 201B is a makeup overlay of a pair of eyebrows (such as shown in FIG. 7C) each eyebrow of eyebrow cosmetic style 201B may be adjusted independently. As another example, if the eyebrow cosmetic style 201B is a lipstick, a blush, and an eyeshadow, the lipstick, the blush, and the eyeshadow may all be adjusted independent of one another.

In some embodiments, the selected eyebrow cosmetic style 201B is adjusted with a plurality of sliders 250A, 250B, 250C . . . 250N. In some embodiments, the selected eyebrow cosmetic style 201B may be adjusted with another mechanism, such as a plurality of presets, manipulation of the selected eyebrow cosmetic style 201B with a touch screen, or otherwise. Once the user is satisfied with the selected eyebrow cosmetic style 201B, the user may then transmit the selected eyebrow cosmetic style 201B as a makeup file to a microblading device 100. In some embodiments, the application may further store the selected and/or adjusted eyebrow cosmetic style 201B as a preset, so that a user can select and/or print the same eyebrow cosmetic style 201B later.

In FIG. 7E, the user 300 applies the selected and/or adjusted eyebrow cosmetic style 201 to a surface with the microblading device 100. In some embodiments, the surface is a face, skin, or hair. In some embodiments, the user 300 may be a second person, where a first person applies the eyebrow cosmetic style to the second person. In some embodiments, the first person may be a trained user, such as in a store or at a makeup counter.

In operation, the microblading device 100 receives the makeup image file of the selected eyebrow cosmetic style 201B. The user 300 may hold the microblading device 100 from handle 135 and move the printer 110 over a surface. In some embodiments, the surface is a face. In some embodiments, the surface is skin or hair. In some embodiments, the surface is a facial feature. As the microblading device 100 is moved over the surface, the microblading device 100 can detect a position and a curvature of the body based on the position sensor and direct the printer to print the eyebrow cosmetic style in a specific location. In some embodiments, the user 300 may direct the microblading device 100 over the surface before selecting, adjusting, and/or printing an eyebrow cosmetic style. In such embodiments, the microblading device 100 may be configured to detect one or more facial features of the user 300 to allow the application to provide a recommendation based on the user's facial features, detect a position of the one or more facial features, or a combination thereof.

In some embodiments, the microblading device 100 can sense its own location on the surface, and automatically apply the one or more eyebrow cosmetic style to the specific location on the surface. In some embodiments, the eyebrow cosmetic style may be manually applied, such as with a button or switch. In this manner, the microblading device 100 prints the eyebrow cosmetic style only in the location desired by the user, based on the eyebrow cosmetic style selected, the adjustments made to the eyebrow cosmetic style, or a combination thereof.

In another aspect, disclosed herein is a method of autonomously applying a eyebrow cosmetic style with the systems described herein. In some embodiments, the method includes selecting the eyebrow cosmetic style from a plurality of eyebrow cosmetic styles, displaying the eyebrow cosmetic style on an image of a body, transferring the eyebrow cosmetic style as an image file to a microblading device, moving the microblading device over the body, and printing the eyebrow cosmetic style onto the body at a specific location based on one or more position sensors on the microblading device.

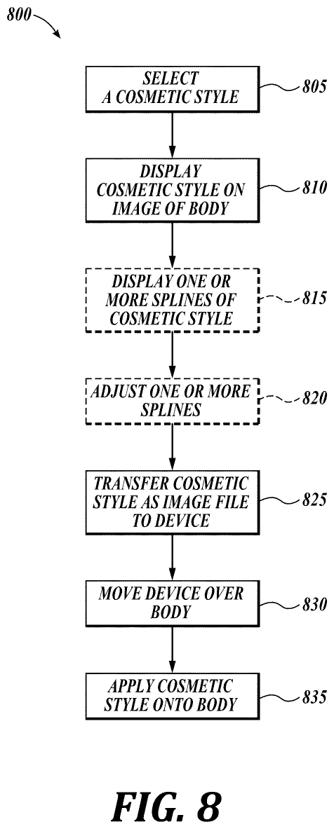

FIG. 8 is an example method 800 of using an example system, in accordance with the present technology.

In block 805, a cosmetic style is selected. In some embodiments, the cosmetic style is selected from a plurality of cosmetic styles. In some embodiments, the cosmetic style is selected as shown in FIGS. 6A, 7A, or a combination thereof. In some embodiments, the cosmetic style is selected from an eyebrow, an eyeshadow, a concealer, a primer, a foundation, a blush, a lipliner, a lipstick, a bronzer, an eyeliner, a freckle pattern, a facial hair, a hair design, a highlighter, or a combination thereof.

In block 810, the cosmetic style is displayed on an image of a body. In some embodiments, the image of the body is a live video feed from a smart device or the printer device. In some embodiments, the cosmetic style is displayed as shown in FIGS. 6B, 7B, or a combination thereof.

Optionally, in block 815, one or more splines of the cosmetic style are displayed (such as shown in FIG. 7C).

Optionally, in block 820, the one or more splines may be adjusted by a user such as by dragging or tapping the spines. In some embodiments, the cosmetic style can be warped, resized, or otherwise modified by adjusting the splines.

In block 825, the cosmetic style is stored as an image file and transferred to a printer device. In some embodiments, the image file is a makeup image file. In some embodiments, the image file may be stored on a smart device application. In some embodiments, the printer device receives the image file of the cosmetic style through a wireless or wired connection.

In block 830, the microblading device is moved over the body. In some embodiments, the microblading device is moved as shown in FIG. 7E. In some embodiments, the microblading device is moved by an operator of the device onto a person. In some embodiments, the microblading device is moved by a user of the microblading device.

In block 835, the cosmetic style is applied onto the body. In some embodiments, the cosmetic style is applied in a location of the body as shown in the image of the body. In some embodiments, the cosmetic style is applied by an applicator as shown in FIGS. 4A-4D. In some embodiments, the cosmetic style is made of one or more cosmetic dyes or inks stored inside one or more reservoirs in the microblading device. In some embodiments, the one or more inks may be applied with one or more microneedles, one or more electrodes, or a combination thereof. In some embodiments, the one or more inks are applied with electroporation.

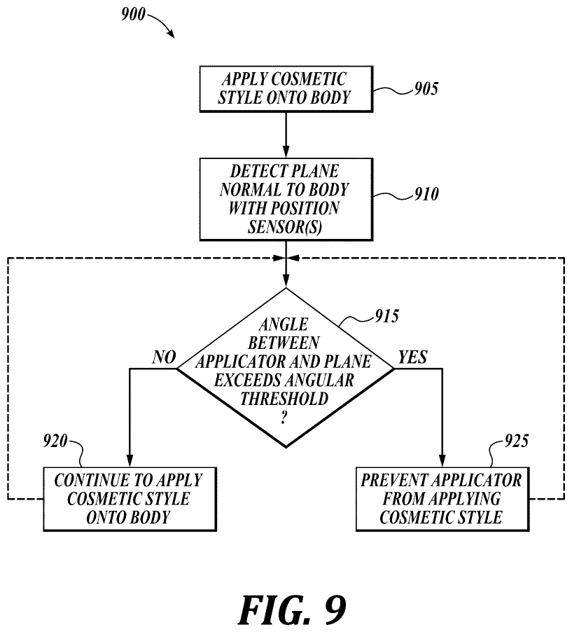

FIG. 9 is another example method 900 of using an example system, in accordance with the present technology. In some embodiments, method 900 occurs directly after method 800.

In block 905, the cosmetic style is applied onto the body. In some embodiments, the cosmetic style is applied in a location of the body as shown in the image of the body. In some embodiments, the cosmetic style is applied by an applicator as shown in FIGS. 4A-4D. In some embodiments, the cosmetic style is made of one or more cosmetic dyes or inks stored inside one or more reservoirs in the microblading device. In some embodiments, the one or more inks may be applied with one or more microneedles, one or more electrodes, or a combination thereof. In some embodiments, the one or more inks are applied with electroporation.

In block 910, a plane normal to a body of the user is detected with one or more position sensors. In some embodiments, the one or more position sensors determine an angle between the applicator of the microblading device and the body of the user.

In decision block 915, the angle between the applicator and the plane normal to the body is compared to an angular threshold. If the angle between the applicator and the plane normal to the body does not exceed the angular threshold, the method proceeds to block 920.

In block 920, the cosmetic style continues to be applied to the body.

Returning to decision block 915, If the angle between the applicator and the plane normal to the body does not exceed the angular threshold, the method proceeds to block 925.

In block 925, the applicator is prevented from applying the cosmetic style.

In some embodiments, the one or more position sensors continuously measure the angle between the applicator and the plane normal to the body and in real time determine whether or not to apply the cosmetic style. In some embodiments, after the cosmetic style is prevented from being applied, the processor prevents the cosmetic style from being applied until the angle between the applicator and the plane normal to the body does not exceed the angular threshold.

It should be understood that all methods 800 and 900 should be interpreted as merely representative. In some embodiments, process blocks of all methods 800 and 900 may be performed simultaneously, sequentially, in a different order, or even omitted, without departing from the scope of this disclosure.

The present application may reference quantities and numbers. Unless specifically stated, such quantities and numbers are not to be considered restrictive, but representative of the possible quantities or numbers associated with the present application. Also, in this regard, the present application may use the term “plurality” to reference a quantity or number. In this regard, the term “plurality” is meant to be any number that is more than one, for example, two, three, four, five, etc. The terms “about,” “approximately,” “near,” etc., mean plus or minus 5% of the stated value. For the purposes of the present disclosure, the phrase “at least one of A, B, and C,” for example, means (A), (B), (C), (A and B), (A and C), (B and C), or (A, B, and C), including all further possible permutations when greater than three elements are listed.

Embodiments disclosed herein may utilize circuitry in order to implement technologies and methodologies described herein, operatively connect two or more components, generate information, determine operation conditions, control an appliance, device, or method, and/or the like. Circuitry of any type can be used. In an embodiment, circuitry includes, among other things, one or more computing devices such as a processor (e.g., a microprocessor), a central processing unit (CPU), a digital signal processor (DSP), an application-specific integrated circuit (ASIC), a field-programmable gate array (FPGA), or the like, or any combinations thereof, and can include discrete digital or analog circuit elements or electronics, or combinations thereof.

An embodiment includes one or more data stores that, for example, store instructions or data. Non-limiting examples of one or more data stores include volatile memory (e.g., Random Access memory (RAM), Dynamic Random Access memory (DRAM), or the like), non-volatile memory (e.g., Read-Only memory (ROM), Electrically Erasable Programmable Read-Only memory (EEPROM), Compact Disc Read-Only memory (CD-ROM), or the like), persistent memory, or the like. Further non-limiting examples of one or more data stores include Erasable Programmable Read-Only memory (EPROM), flash memory, or the like. The one or more data stores can be connected to, for example, one or more computing devices by one or more instructions, data, or power buses.

In an embodiment, circuitry includes a computer-readable media drive or memory slot configured to accept signal-bearing medium (e.g., computer-readable memory media, computer-readable recording media, or the like). In an embodiment, a program for causing a system to execute any of the disclosed methods can be stored on, for example, a computer-readable recording medium (CRMM), a signal-bearing medium, or the like. Non-limiting examples of signal-bearing media include a recordable type medium such as any form of flash memory, magnetic tape, floppy disk, a hard disk drive, a Compact Disc (CD), a Digital Video Disk (DVD), Blu-Ray Disc, a digital tape, a computer memory, or the like, as well as transmission type medium such as a digital and/or an analog communication medium (e.g., a fiber optic cable, a waveguide, a wired communications link, a wireless communication link (e.g., transmitter, receiver, transceiver, transmission logic, reception logic, etc.). Further non-limiting examples of signal-bearing media include, but are not limited to, DVD-ROM, DVD-RAM, DVD+RW, DVD-RW, DVD-R, DVD+R, CD-ROM, Super Audio CD, CD-R, CD+R, CD+RW, CD-RW, Video Compact Discs, Super Video Discs, flash memory, magnetic tape, magneto-optic disk, MINIDISC, non-volatile memory card, EEPROM, optical disk, optical storage, RAM, ROM, system memory, web server, or the like.

The detailed description set forth above in connection with the appended drawings, where like numerals reference like elements, are intended as a description of various embodiments of the present disclosure and are not intended to represent the only embodiments. Each embodiment described in this disclosure is provided merely as an example or illustration and should not be construed as preferred or advantageous over other embodiments. The illustrative examples provided herein are not intended to be exhaustive or to limit the disclosure to the precise forms disclosed. Similarly, any steps described herein may be interchangeable with other steps, or combinations of steps, in order to achieve the same or substantially similar result. Generally, the embodiments disclosed herein are non-limiting, and the inventors contemplate that other embodiments within the scope of this disclosure may include structures and functionalities from more than one specific embodiment shown in the figures and described in the specification.

In the foregoing description, specific details are set forth to provide a thorough understanding of exemplary embodiments of the present disclosure. It will be apparent to one skilled in the art, however, that the embodiments disclosed herein may be practiced without embodying all the specific details. In some instances, well-known process steps have not been described in detail in order not to unnecessarily obscure various aspects of the present disclosure. Further, it will be appreciated that embodiments of the present disclosure may employ any combination of features described herein.

The present application may include references to directions, such as “vertical,” “horizontal,” “front,” “rear,” “left,” “right,” “top,” and “bottom,” etc. These references, and other similar references in the present application, are intended to assist in helping describe and understand the particular embodiment (such as when the embodiment is positioned for use) and are not intended to limit the present disclosure to these directions or locations.

The present application may also reference quantities and numbers. Unless specifically stated, such quantities and numbers are not to be considered restrictive, but exemplary of the possible quantities or numbers associated with the present application. Also, in this regard, the present application may use the term “plurality” to reference a quantity or number. In this regard, the term “plurality” is meant to be any number that is more than one, for example, two, three, four, five, etc. The term “about,” “approximately,” etc., means plus or minus 5% of the stated value. The term “based upon” means “based at least partially upon.”

The principles, representative embodiments, and modes of operation of the present disclosure have been described in the foregoing description. However, aspects of the present disclosure, which are intended to be protected, are not to be construed as limited to the particular embodiments disclosed. Further, the embodiments described herein are to be regarded as illustrative rather than restrictive. It will be appreciated that variations and changes may be made by others, and equivalents employed, without departing from the spirit of the present disclosure. Accordingly, it is expressly intended that all such variations, changes, and equivalents fall within the spirit and scope of the present disclosure as claimed.

While illustrative embodiments have been illustrated and described, it will be appreciated that various changes can be made therein without departing from the spirit and scope of the invention.

We claim:

1. A microblading device, comprising:
at least one position sensor configured to measure a position of the microblading device;
one or more reservoirs, each configured to hold one or more inks;
an applicator configured to apply the one or more inks into skin;
a power source;
at least one pressure sensor configured to measure a pressure of the applicator; and
a processor configured to:
receive an image file comprising an eyebrow cosmetic style,
detect the position and a curvature of a body based on the position sensor,
direct the applicator to apply the eyebrow cosmetic style in a specific location with the one or more inks, and
modulate the pressure of the applicator based on the image file.

2. The microblading device of claim 1, wherein the applicator is one or more microneedles.

3. The microblading device of claim 2, wherein the one or more microneedles are arranged in an array.

4. The microblading device of claim 2, wherein the device further comprises a motor configured to reciprocate the microneedle.

5. The microblading device of claim 1, wherein the applicator is one or more electrodes configured to apply the one or more inks with electroporation.

6. The microblading device of claim 5, wherein the microblading device further comprises a pulse generator configured to deliver an electrical current through the one or more electrodes.

7. The microblading device of claim 1, wherein the at least one position sensor is configured to detect a plane normal to the body, and wherein the processor is further configured to prevent the applicator from applying the eyebrow cosmetic style when an angle between the applicator and the plane normal to the body exceeds an angular threshold.

8. The microblading device of claim 1, wherein the processor is further configured to modulate a penetration depth of the applicator based on the eyebrow cosmetic style.

9. The microblading device of claim 8, wherein the penetration depth ranges from about 0.2 mm to about 2 mm into skin.

10. A system for applying cosmetics, comprising:
the microblading device of claim 1; and
a smart device comprising an application configured to:
select the eyebrow cosmetic style from a plurality of eyebrow cosmetic styles;
display the eyebrow cosmetic style on an image of a body; and
transmit the eyebrow cosmetic style as an image file to the microblading device.

11. The system of claim 10, wherein the image file further comprises one or more pressure metrics, one or more penetration depths, or a combination thereof.

12. The system of claim 10, wherein the eyebrow cosmetic style comprises one or more eyebrow mimetics.

13. The system of claim 12, wherein the one or more eyebrow mimetics comprises a randomization factor, one or more hair-like strokes, one or more fractals, or a combination thereof.

14. The system of claim 12, wherein the application is further configured to:
display one or more splines of the eyebrow cosmetic style on the image of the body.

15. A method of using a microblading device, the microblading device comprising at least one position sensor, one or more reservoirs, each configured to hold one or more inks, an applicator configured to apply the one or more inks, at least one pressure sensor, a power source; and a processor, the method comprising:
selecting an eyebrow cosmetic style from a plurality of eyebrow cosmetic styles;
displaying the eyebrow cosmetic style on an image of a body;
transferring the eyebrow cosmetic style as an image file to the microblading device, wherein the image file further comprises one or more pressure metrics;
moving the microblading device over the body;
applying the eyebrow cosmetic style onto the body at a specific location based on one or more position sensors on the microblading device; and
modulating a pressure of the applicator based on the one or more pressure metrics.

16. The method of claim 15, wherein the method further comprises recommending the eyebrow cosmetic style based on a trending eyebrow cosmetic style, a color of a user's hair, skin, or lips, a past eyebrow cosmetic style selected by the user, a shape of the user's eyes, eyebrows, nose, lips, cheeks, or forehead, a location of the user, a color of the user's clothing or a combination thereof.

17. The method of claim 15, further comprising adjusting the eyebrow cosmetic style, wherein adjusting the eyebrow cosmetic style comprises changing a color, a size, an angle, a length, a width, a hair size, a pattern, a position, a location of the eyebrow cosmetic style, or a combination thereof.

18. The method of claim 15, wherein the microblading device applies the eyebrow cosmetic style with one or more microneedles, one or more electrodes, or a combination thereof.

19. The method of claim 15, further comprising:
detecting a plane normal to the body with the one or more position sensors; and
preventing the applicator from applying the eyebrow cosmetic style when an angle between the applicator and the plane normal to the body exceeds an angular threshold.

20. The method of claim 15, further comprising:
displaying one or more splines of the eyebrow cosmetic style on the image of the body; and
adjusting the one or more splines in response to user input.
